(19) United States
(12) Patent Application Publication
(10) Pub. No.: US 2003/0211126 A1
(43) Pub. Date: Nov. 13, 2003

(54) Composition for topically delivering vitamin c

(76) Inventors: Richard E Fitzpatrick, Sante Fe, CA (US); John A Garruto, Encinitas, CA (US)

Correspondence Address: David A Farah, Sheldon & Mak, 9th Floor, 225 South Lake Avenue, Pasadena, CA, 91101, US

(21) Appl. No.: 10/332,725

(22) PCT Filed: Jul. 12, 2001
(86) PCT No.: PCT/US01/21949

(30) Foreign Application Priority Data
Jul. 13, 2000 (US) 09614691

(51) Int. Cl. A61K 31/375; A61K 7/00

(52) U.S. Cl. 424/401; 514/474

(57) ABSTRACT

A composition for the topical application of vitamin C consisting essentially of one or more lipid-soluble forms of vitamin C, one or more water-soluble forms of vitamin C and one or more alpha hydroxylated acids. The composition can also include an anhydrous gel, ethoxydiglycol, a lipid-soluble analog of pro-vitamin B-5, alpha-Bisabolol, and one or more forms of vitamin E. A composition for the topical application of vitamin C consisting of one or more lipid-soluble forms of vitamin C, one or more water-soluble forms of vitamin C and one or more alpha hydroxylated acids. The composition can also include an anhydrous gel, ethoxydiglycol, a lipid-soluble analog of pro-vitamin B-5, alpha-Bisabolol, and one or more forms of vitamin E.

### CROSS-REFERENCE TO RELATED APPLICATION

This application is a national phase filing under 35 U.S.C. §371 of PCT Application PCT/US01/21949, filed Jul. 12, 2001 and titled “Composition for the Topical Delivery of Vitamin C”; which claims priority from U.S. patent application Ser. No. 09/614,691, filed Jul. 13, 2000 and titled “Composition for the Topical Delivery of Vitamin C,” the contents of which are incorporated herein by reference in their entirety.

### BACKGROUND

L-ascorbic acid, generally known as vitamin C, is an essential requirement in the diets of primates, including humans. Vitamin C is necessary for the normal synthesis of collagen. Deficiencies in vitamin C leads to impairment of peptidyl hydroxylation of procollagen and a reduction in collagen formation and collagen secretion by connective tissue. The manifestations of vitamin C deficiency include fragile capillaries leading to hemorrhages and poor wound healing. Additionally, vitamin C deficiency leads to atrophy of the dermis layer of the skin which causes wrinkles and wounds.

Vitamin C can be taken orally. However, it is advantageous in some conditions to apply vitamin C topically to a site where connective tissue formation is needed. These conditions include evidential signs of aging such as wrinkling caused by environmental exposure such as actinic aging caused by exposure to ultraviolet radiation from the sun.

Most currently known compositions for the topical application of vitamin C contain only water-soluble analogs of vitamin C, such as L-ascorbic acid, in an aqueous vehicle that delivers the vitamin C immediately at the application site. Some of these compositions cause a significant incidence of irritation at the application site. Additionally, many forms of vitamin C, such as L-ascorbic acid, used in these preparations are very unstable in an aqueous vehicle and do not penetrate intact skin well.

Therefore, it would be useful to have a composition for the topical application of vitamin C which can be used to deliver vitamin C to a specific site over an extended period of time. It would also be useful to have a composition for the topical application of vitamin C, which has a lower incidence of irritation at the application site. Further, it would be useful to have a composition for the topical application of vitamin C in which the form of vitamin C is more stable.

### SUMMARY

In one embodiment, there is provided a composition for the topical application of vitamin C. The composition comprises one or more lipid-soluble forms of vitamin C, one or more water-soluble forms of vitamin C and one or more alpha hydroxylated acids. Preferably, the composition also comprises an anhydrous gel. In preferred embodiments, the composition additionally comprises one or more substances selected from the group consisting of ethoxydiglycol, a lipid-soluble analog of pro-vitamin B-5, alpha-Bisabolol, and a form of vitamin E.

In another preferred embodiment, there is provided a composition for the topical application of vitamin C consisting essentially of one or more lipid-soluble forms of vitamin C, one or more water-soluble forms of vitamin C and one or more alpha hydroxylated acids. In a preferred embodiment, there is provided a composition consisting essentially of one or more lipid-soluble forms of vitamin C, one or more water-soluble forms of vitamin C, one or more alpha hydroxylated acids and an anhydrous gel. Also provided is a composition for the topical application of vitamin C consisting essentially of one or more lipid-soluble forms of vitamin C, one or more water-soluble forms of vitamin C, one or more alpha hydroxylated acids, an anhydrous gel, and one or more substances selected from the group consisting of ethoxydiglycol, a lipid-soluble analog of pro-vitamin B-5, alpha-Bisabolol, and a form of vitamin E.

In another preferred embodiment, there is provided a composition for the topical application of vitamin C consisting of one or more lipid-soluble forms of vitamin C, one or more water-soluble forms of vitamin C and one or more alpha hydroxylated acids. In another preferred embodiment, there is provided a composition consisting of one or more lipid-soluble forms of vitamin C, one or more water-soluble forms of vitamin C, one or more alpha hydroxylated acids and an anhydrous gel. In another preferred embodiment, there is provided a composition consisting of one or more lipid-soluble forms of vitamin C, one or more water-soluble forms of vitamin C, one or more alpha hydroxylated acids, an anhydrous gel and one or more substances selected from the group consisting of ethoxydiglycol, a lipid-soluble analog of pro-vitamin B-5, alpha-Bisabolol, and a form of vitamin E.

### DESCRIPTION

According to one embodiment of the present invention, there is provided a composition for the topical application of vitamin C. The composition supplies usable vitamin C to a specific area to promote collagen formation which thickens the dermis, thereby diminishing fine skin lines and wrinkles, among other benefits. The composition comprises one or more water-soluble forms of vitamin C and one or more lipid-soluble forms of vitamin C. The composition also comprises one or more alpha hydroxylated acids. The use of both a lipid-soluble form and a water-soluble form of vitamin C in the same composition synergistically increases total surface penetration of vitamin C. The use of the at least one alpha hydroxylated acid additionally enhances the skin penetration of the vitamin C analogs.

All amounts disclosed herein are given in weight percent of the total weight of the composition.

The water-soluble form of vitamin C included in the composition provides immediate antioxidant effects to the application site. Suitable water-soluble forms of vitamin C for use in the present composition include L-ascorbic acid or mineral salts of ascorbic acid, such as sodium ascorbate, potassium ascorbate or magnesium ascorbate. In a preferred embodiment, the water-soluble forms of vitamin C for use in the present composition is ascorbic acid, ultra-fine available from Roche Vitamins, Inc., Parsippany, N.J. US. In a preferred embodiment, the one or more water-soluble forms of vitamin C is present in an amount of between about 3% and about 20% by weight of the total weight of the composition, and particularly between about 8% and about 15% by weight of the total weight of the composition.

The lipid-soluble form of vitamin C included in the composition increases the penetration of the vitamin C component of the composition and is converted to ascorbic acid within the dermis. Further, the lipid-soluble form is more stable than the water-soluble form of vitamin C and helps insure that the vitamin C component of the present invention remains in a usable form during storage.

Suitable lipid-soluble forms of vitamin C for use in the present composition include an ester of vitamin C. In a preferred embodiment, the lipid-soluble form of vitamin C is tetrahexyldecyl ascorbate available from Barnet Products Corp., Englewood Cliffs, N.J. US under the name BV-OSC™. In a less preferred embodiment, the lipid-soluble form of vitamin C is ascorbyl palmitate available from Roche Vitamins, Inc., Parsippany, N.J. US. In a preferred embodiment, the one or more lipid-soluble forms of vitamin C is present in an amount of between about 2% and about 20% by weight of the total weight of the composition, and particularly between about 5% and about 10% by weight of the total weight of the composition.

The composition of the present invention also comprises one or more alpha hydroxylated acids which functions as a skin penetration enhancer. The alpha hydroxylated acid increases skin surface cell exfoliation, thereby increasing the penetration of the vitamin C into the skin. The alpha hydroxylated acid or acids also decrease fine skin lines and wrinkles and increases the moisturizing effect of the composition.

In one embodiment, alpha hydroxylated acid has between about 8 and about 12 carbon groups. Suitable alpha hydroxylated acids for use in the present composition include alpha hydroxylated acids selected from the group consisting of 1) alpha hydroxylated caprylic acid (Lipoamidroxy 8™, item #85712), 2) alpha hydroxylated capric acid (Lipoamidroxy 10™, item #85715), and 3) alpha hydroxylated lauric acid (Lipoamidroxy 12™, item #85714), available from Tri-K Industries, Northvale, N.J. US. Additionally, more than one alpha hydroxylated acid can be used together. In a preferred embodiment, the composition comprises a total amount of between about 0.5% and about 10% of the alpha hydroxylated acid or alpha hydroxylated acids, and particularly between about 1% and about 5% of the alpha hydroxylated acid or alpha hydroxylated acids.

The composition of the present invention preferably also comprises an anhydrous gel as a carrier for the water-soluble analog form of vitamin C and the lipid-soluble analog form of vitamin C and the alpha hydroxylated acid. A suitable anhydrous gel is a combination of cyclic silicone and polysilicone. The anhydrous gel helps stabilize the water-soluble form of vitamin C due to the anhydrous nature of the gel. The anhydrous gel further acts as a transdermal patch and allows for the release of vitamin C over a period of time when applied topically.

In a preferred embodiment, the anhydrous gel for use in the present composition is Gransil GCM-5™, CAS #TSRN-8450, available from Grant Industries, Elmwood Park, N.J. US. In another preferred embodiment, the anhydrous gel for use in the present composition is a combination of super white petrolatum, CAS #8009-03-8, available from Penreco Chemical, Los Angeles, Calif. US and lipid soluble esters such as cetyl octanoate, CAS #59130-69-7 or diisopropyl fumarate, CAS #113431-53-1, available from Scher Chemicals, West Clifton, N.J. US, or a combination of cetyl octanoate or diisopropyl fumarate.

In a preferred embodiment, the composition comprises at least about 65% anhydrous gel. In another preferred embodiment, the composition comprises between about 70% and about 90% anhydrous gel. In a particularly preferred embodiment, the composition comprises about 80% anhydrous gel. The composition can contain, for example, at least about 60% petrolatum, and at least about 5% cetyl octanoate or diisopropyl fumarate, or at least about 5% of a combination of cetyl octanoate or diisopropyl fumarate.

The composition can also comprise one or more other skin penetration enhancer, such as ethoxydiglycol (Transcutol™), available from Gattefosse Corporation, Westwood, N.J. US. In a preferred embodiment, the composition comprises between about 0.5% and about 10% of the ethoxydiglycol, and particularly between about 1% and about 5% of the ethoxydiglycol.

The composition of the present invention can also comprise one or more additional substance such as a moisturizer or an anti-irritant, or can comprise more than one additional substance such as a combination of one or more moisturizer and one or more anti-irritant. For example, the composition preferably includes a lipid-soluble analog of pro-vitamin B-5 such as phytantriol, CAS #7456-64-7 (3,7,11,15-tetramethyl-1,2,3-hexadecanetriol) available from BASF Nutrition and Cosmetics, Mount Olive, N.J. US. Phytantriol is both a moisturizer and an anti-irritant. In a preferred embodiment, the composition comprises between about 0.1% and about 2% of the lipid-soluble analog of pro-vitamin B-5, and particularly between about 0.2% and about 1% of the lipid-soluble analog of pro-vitamin B-5.

In another preferred embodiment, the composition comprises (1-Methyl-4(1,5-dimethyl-1-hydroxyhex-4(5)-enyl)-cyclohexen-1; 6-Methyl-2-(4-methyl-3-cyclohexen-1-yl)-5-hepten-2-oligonucleotides) (alpha-Bisabolol) available from BASF Nutrition and Cosmetics, Mount Olive, N.J. US as an anti-irritant. In a preferred embodiment, the composition comprises between about 0.1% and about 2% of the alpha-Bisabolol, and particularly between about 0.2% and about 1% of the alpha-Bisabolol.

In another preferred embodiment, the composition comprises one or more antioxidants in addition to the vitamin C analogs such as a form of tocopherol, generally known as vitamin E. The vitamin E serves to decrease the irritation caused by application of the water-soluble form of vitamin C. In a particularly preferred embodiment, the composition comprises a form of vitamin E selected from the group consisting of tocopheryl acetate and Coviox T-50™. Coviox T-50™ is a blend of mixed, naturally occurring tocopherols which is available from Cognis Corporation, Cincinnati, Ohio US. In another preferred embodiment, the composition comprises tocopheryl acetate, available from Nutrition and Cosmetics, Mount Olive, N.J. US.

In a preferred embodiment, the composition comprises between about 0.1% and about 2% of the additional antioxidant, and particularly between about 0.2% and about 1% of the additional antioxidant. In another preferred embodiment, the composition comprises both Coviox T-50™ and tocopheryl acetate.

### EXAMPLE I

### Making a Composition for the Topical Application of Vitamin C

According to one embodiment of the present invention, a composition for the topical application of vitamin C comprises the following substances:

| | PERCENT |
|---|---|
| | WEIGHT OF |
| SUBSTANCE | TOTAL WEIGHT |
| anhydrous gel ((77 percent super white petrolatum | 82 |
| and 5 percent diisopropyl fumarate) or Gransil | |
| GCM-5 ™) | |
| water-soluble analog of vitamin C | 10 |
| (ascorbic acid, ultra-fine) | |
| lipid-soluble analog of vitamin C | 5 |
| (tetrahexyldecyl ascorbate) (BV-OSC ™) | |
| alpha hydroxylated acid (Lipoamidroxy 12 ™) | 1 |
| skin penetration enhancer | 1 |
| (ethoxydiglycol) (Transcutol ™) | |
| lipid-soluble analog of pro-vitamin B-5 (phytantriol) | 0.3 |
| vitamin E (tocopheryl acetate) | 0.3 |
| (1-Methyl-4(1,5-dimethyl-1-hydroxyhex-4(5)-enyl)- | 0.2 |
| cyclohexen-1; 6-Methyl-2-(4-methyl-3-cyclohexen- | |
| 1-yl)-5-hepten-2-oligonucleotides) (alpha-Bisabolol) | |
| vitamin E (Coviox T-50 ™) | 0.2 |

The composition is preferably made by adding the anhydrous gel (super white petrolatum and diisopropyl fumarate, or Gransil GCM-5™) to a mixing tank, and then slowly adding the water-soluble analog of vitamin C (ascorbic acid, ultra-fine) and alpha hydroxylated acid (Lipoamidroxy 12™) to the anhydrous gel, while mixing carefully to avoid excess air entrapment. Next, the remaining substances are added in the following order: 1) skin penetration enhancer (ethoxydiglycol) (Transcutol™); 2) lipid-soluble analog of vitamin C (tetrahexyldecyl ascorbate) (BV-OSC™); 3) lipid-soluble analog of pro-vitamin B-5 (phytantriol); 4) vitamin E (tocopheryl acetate); 5) vitamin E (Coviox T-50™); and 6) (1-Methyl-4(1,5-dimethyl-1-hydroxyhex-4(5)-enyl)-cyclohexen-1; 6-Methyl-2-(4-methyl-3-cyclohexen-1-yl)-5-hepten-2-oligonucleotides) (Bisabolol), insuring that each substance is completely dispersed before adding the next ingredient.

### EXAMPLE II

### Use of a Composition for the Topical Application of Vitamin C

The composition to the present invention can be used by virtually all persons who do not have overt skin disease or open wounds. The composition to the present invention is particularly useful for persons who have skin with excessive sun exposure, have a history of sun sensitivity, pigmentation abnormalities, precancerous skin lesions and who have a fair complexion.

The composition of the present invention is preferably applied to the area to be treated each morning before applying other skin care products, though skin care products with an acidic pH can be applied prior to the application of the composition to the present invention, if desired.

The composition to the present invention should be applied for a minimum of about one year, though positive effects can be seen with use of less than one year. The treatment time should be maintained for at least one year to one and one half years, but can be continued indefinitely depending on the needs and results of the individual user.

Although the present invention has been discussed in considerable detail with reference to certain preferred embodiments, other embodiments are possible. Therefore, the scope of the appended claims should not be limited to the description of preferred embodiments contained herein.

What is claimed is:

1. A composition for the topical application of vitamin C consisting essentially of one or more lipid-soluble forms of vitamin C, one or more water-soluble forms of vitamin C and one or more alpha hydroxylated acids.

2. The composition of claim 1, where the one or more water-soluble forms of vitamin C is L-ascorbic acid.

3. The composition of claim 1, where the one or more water-soluble forms of vitamin C is present in an amount of between about 3% and about 20% by weight of the total weight of the composition.

4. The composition of claim 1, where the one or more water-soluble forms of vitamin C is present in an amount of between about 8% and about 15% by weight of the total weight of the composition.

5. The composition of claim 1, where the one or more lipid-soluble forms of vitamin C is tetrahexyldecyl ascorbate.

6. The composition of claim 1, where the one or more lipid-soluble forms of vitamin C is present in an amount of between about 2% and about 20% by weight of the total weight of the composition.

7. The composition of claim 1, where the one or more lipid-soluble forms of vitamin C is present in an amount of between about 5% and about 10% by weight of the total weight of the composition.

8. The composition of claim 1, where the one or more alpha hydroxylated acids has between about 8 and about 12 carbon groups.

9. The composition of claim 1, where the one or more alpha hydroxylated acids is selected from the group consisting of alpha hydroxylated caprylic acid, alpha hydroxylated capric acid and alpha hydroxylated lauric acid.

10. The composition of claim 1, where the one or more alpha hydroxylated acids is present in an amount of between about 0.5% and about 10% by weight of the total weight of the composition.

11. The composition of claim 1, where the one or more alpha hydroxylated acids is present in an amount of between about 1% and about 5% by weight of the total weight of the composition.

12. A composition consisting essentially of the composition of claim 1, and an anhydrous gel.

13. The composition of claim 12, where the anhydrous gel is a combination of cyclic silicone and polysilicone.

14. The composition of claim 12, where the anhydrous gel is a combination of super white petrolatum and cetyl octanoate.

15. The composition of claim 12, where the anhydrous gel is a combination of super white petrolatum and diisopropyl fumarate.

16. The composition of claim 12, where the anhydrous gel is present in an amount of at least about 65% by weight of the total weight of the composition.

17. The composition of claim 12, where the anhydrous gel is present in an amount of between about 70% and about 90% by weight of the total weight of the composition.

18. A composition consisting essentially of the composition of claim 1, and one or more substances selected from the group consisting of ethoxydiglycol, a lipid-soluble analog of pro-vitamin B-5, alpha-Bisabolol, and one or more forms of vitamin E.

19. A composition consisting essentially of the composition of claim 12, and one or more substances selected from the group consisting of ethoxydiglycol, a lipid-soluble analog of pro-vitamin B-5, alpha-Bisabolol, and one or more forms of vitamin E.

20. A composition for the topical application of vitamin C consisting of one or more lipid-soluble forms of vitamin C, one or more water-soluble forms of vitamin C and one or more alpha hydroxylated acids.

21. The composition of claim 20, where the one or more water-soluble forms of vitamin C is L-ascorbic acid.

22. The composition of claim 20, where the one or more water-soluble forms of vitamin C is present in an amount of between about 3% and about 20% by weight of the total weight of the composition.

23. The composition of claim 20, where the one or more water-soluble forms of vitamin C is present in an amount of between about 8% and about 15% by weight of the total weight of the composition.

24. The composition of claim 20, where the one or more lipid-soluble forms of vitamin C is tetrahexyldecyl ascorbate.

25. The composition of claim 20, where the one or more lipid-soluble forms of vitamin C is present in an amount of between about 2% and about 20% by weight of the total weight of the composition.

26. The composition of claim 20, where the one or more lipid-soluble forms of vitamin C is present in an amount of between about 5% and about 10% by weight of the total weight of the composition.

27. The composition of claim 20, where the one or more alpha hydroxylated acids has between about 8 and about 12 carbon groups.

28. The composition of claim 20, where the one or more alpha hydroxylated acids is selected from the group consisting of alpha hydroxylated caprylic acid, alpha hydroxylated capric acid and alpha hydroxylated lauric acid.

29. The composition of claim 20, where the one or more alpha hydroxylated acids is present in an amount of between about 0.5% and about 10% by weight of the total weight of the composition.

30. The composition of claim 20, where the one or more alpha hydroxylated acids is present in an amount of between about 1% and about 5% by weight of the total weight of the composition.

31. A composition consisting of the composition of claim 20, and an anhydrous gel.

32. The composition of claim 31, where the anhydrous gel is a combination of cyclic silicone and polysilicone.

33. The composition of claim 31, where the anhydrous gel is a combination of super white petrolatum and cetyl octanoate.

34. The composition of claim 31, where the anhydrous gel is a combination of super white petrolatum and diisopropyl fumarate.

35. The composition of claim 31, where the anhydrous gel is present in an amount of at least about 55% by weight of the total weight of the composition.

36. The composition of claim 31, where the anhydrous gel is present in an amount of between about 70% and about 90% by weight of the total weight of the composition.

37. A composition consisting of the composition of claim 20, and one or more substances selected from the group consisting of ethoxydiglycol, a lipid-soluble analog of pro-vitamin B-5, alpha-Bisabolol, and one or more forms of vitamin E.

38. A composition consisting of the composition of claim 31, and one or more substances selected from the group consisting of ethoxydiglycol, a lipid-soluble analog of pro-vitamin B-5, alpha-Bisabolol, and one or more forms of vitamin E.

39. A composition comprising the composition of claim 20, and one or more substances selected from the group consisting of ethoxydiglycol, a lipid-soluble analog of pro-vitamin B-5, alpha-Bisabolol, and one or more forms of vitamin E.

40. A composition comprising the composition of claim 31, and one or more substances selected from the group consisting of ethoxydiglycol, a lipid-soluble analog of pro-vitamin B-5, alpha-Bisabolol, and one or more forms of vitamin E.
